(19) United States
(12) Patent Application Publication
(10) Pub. No.: US 2022/0040141 A1
(43) Pub. Date: Feb. 10, 2022

(54) ULTRAPURE PHENOL COMPOSITIONS

(71) Applicant: Blue Harvest, LLC, Chicago, IL (US)

(72) Inventors: Steven L. DENENBERG, Chicago, IL (US); Bradley J. COLE, Chicago, IL (US); Nikola N. TANEV, Chicago, IL (US)

(73) Assignee: Blue Harvest, LLC, Chicago, IL (US)

(21) Appl. No.: 17/281,359

(22) PCT Filed: Oct. 1, 2019
(86) PCT No.: PCT/US2019/054077

§ 371 Date: Mar. 30, 2021

Related U.S. Application Data
(60) Provisional application No. 62/739,568, filed on Oct. 1, 2018.

(51) Int. Cl. A61K 31/352; A61K 47/14; A61K 31/05; A61K 36/23; A61K 36/9066; A61K 36/9068; A61K 36/53; A61K 36/35; A61K 36/28; A61K 31/015; A61K 36/76; A61K 36/54; A61K 9/00

(52) U.S. Cl. CPC A61K 31/352; A61K 47/14; A61K 31/05; A61K 36/23; A61K 36/9066; A61K 36/9068; A61K 9/0053; A61K 36/35; A61K 36/28; A61K 31/015; A61K 36/76; A61K 36/54; A61K 36/53

(57) ABSTRACT

A composition can include a cannabinoid and caprylic acid triglyceride.

### CLAIM OF PRIORITY

This application claims priority to U.S. Provisional Patent No. 62/739,568, filed Oct. 1, 2018, which is incorporated by reference in its entirety.

### FIELD OF THE INVENTION

The invention relates to compositions including a triglyceride and a purified phenol compounds and derivatives thereof.

### BACKGROUND

Cannabidiol (CBD) is a cannabinoid that can have beneficial health effects in mammals when appropriately administered.

### SUMMARY

In general, a composition including a cannabinoid and a pure triglyceride can have important health effects on mammals. The cannabinoid can be a pure cannabinoid.

In another aspect, a composition can consisting essentially of a cannabinoid, caprylic acid triglyceride and, optionally, a plant based additive. The composition can include 1, 2, 3, 4 or 5 different plant based additives. The cannabinoid can be pure, for example, at least 80%, at least 85%, at least 95% purity, at least 97% purity, at least 98% purity, or at least 99% purity. The caprylic acid triglyceride can contain at least 90%, at least 95%, or at least 98% of a caprylic acid component.In another aspect, a method of delivering a composition described herein to a mammal can include orally dosing the composition to the mammal. In certain circumstances, orally dosing the composition can include spraying the composition onto at least a portion of the tongue of the mammal, spraying the composition under at least a portion of the tongue of the mammal or depositing a drop of the composition onto at least an oral mucosal membrane of the mammal.

The cannabinoid can be a pure cannabinoid. For example, the cannabinoid can be of at least 80%, at least 85%, at least 95% purity, at least 97% purity, at least 98% purity, or at least 99% purity. The pure cannabinoid can be cannabidiol.

The pure triglyceride is a triglyceride containing glycerol and substantially a single chain length carboxylic acid. By substantially a single chain length, less than 10%, preferably less than 5% or more preferably less than 2% of the carboxylic acid component of the triglyceride is of a chain length different from the predominant chain length of the triglyceride. For example, the pure triglyceride can be a C8 triglyceride, such as caprylic acid triglyceride containing less than 10%, less than 5% or less than 2% of a capric acid component and at least 90%, at least 95%, or at least 98% of a caprylic acid component.

In one aspect, a composition can include a pure cannabidiol and caprylic acid triglyceride. In certain circumstances, the composition can include 0.01 wt % to 10 wt %, cannabidiol and 90 wt % to 99.99 wt % caprylic acid triglyceride. For example, the composition can include 90 wt % to 99.99 wt % caprylic acid triglyceride. In other circumstances, the composition can include 10 wt % to 40 wt %, cannabidiol and 60 wt % to 90 wt % caprylic acid triglyceride. In certain circumstances, the amount of cannabidiol in the composition can be 0.01 wt %, 0.05 wt %, 0.1 wt %, 0.2 wt %, 0.3 wt %, 0.4 wt %, 0.5 wt %, 1 wt %, 2 wt %, 5 wt %, 10 wt %, 15 wt %, 20 wt %, 25 wt %, 30 wt %, 35 wt %, or 40 wt %.

In certain circumstances, the weight ratio of cannabinoid to triglyceride in the composition can be between 1:500 and 1:10, between 1:250 and 1:20, or between 1:100 and 1:50.

In certain circumstances, the composition can include a cannabinoid. For example, the composition can include a combination of cannabinoids. The cannabinoid can be in combination with the cannabidiol or can replace some portion of the cannabidiol. The cannabinoid can be of at least 80%, at least 85%, at least 95% purity, at least 97% purity, at least 98% purity, or at least 99% purity. The cannabinoid can be cannabinol, cannabidiol, dronabinol (delta-9-tetrahydrocannabinol), delta-8-tetrahydrocannabinol, 11-hydroxy-tetrahydrocannabinol, 11-hydroxy-delta-9-tetrahydrocannabinol, levonantradol, delta-11-tetrahydrocannabinol, or tetrahydrocannabivarin. In certain circumstances, the cannabinoid can be tetrahydrocannabinol. The cannabinoid is an example of a purified phenol compound or derivative thereof.

In another aspect, a composition can consisting essentially of tetrahydrocannabinol, caprylic acid triglyceride and, optionally, a plant based additive. The composition can include 1, 2, 3, 4 or 5 different plant based additives. The tetrahydrocannabinol and caprylic acid triglyceride can be pure.

The tetrahydrocannabinol can be a pure tetrahydrocannabinol. For example, the cannabidiol can be of at least 80%, at least 85%, at least 95% purity, at least 97% purity, at least 98% purity, or at least 99% purity.

The pure triglyceride is a triglyceride containing glycerol and substantially a single chain length carboxylic acid. By substantially a single chain length, less than 10%, preferably less than 5% or more preferably less than 2% of the carboxylic acid component of the triglyceride is of a chain length different from the predominant chain length of the triglyceride. For example, the pure triglyceride can be a C8 triglyceride, such as caprylic acid triglyceride containing less than 2% of a capric acid component and at least 98% a caprylic acid component.

In one aspect, a composition can include a pure tetrahydrocannabinol and caprylic acid triglyceride. In certain circumstances, the composition can include 0.01 wt % to 10 wt %, tetrahydrocannabinol and 90 wt % to 99.99 wt % caprylic acid triglyceride. For example, the composition can include 90 wt % to 99.99 wt % caprylic acid triglyceride. In other circumstances, the composition can include 10 wt % to 40 wt %, tetrahydrocannabinol and 60 wt % to 90 wt % caprylic acid triglyceride. In certain circumstances, the amount of tetrahydrocannabinol in the composition can be 0.01 wt %, 0.05 wt %, 0.1 wt %, 0.2 wt %, 0.3 wt %, 0.4 wt %, 0.5 wt %, 1 wt %, 2 wt %, 5 wt %, 10 wt %, 15 wt %, 20 wt %, 25 wt %, 30 wt %, 35 wt %, or 40 wt %.

In certain circumstances, the weight ratio of tetrahydrocannabinol to triglyceride in the composition can be between 1:500 and 1:10, between 1:250 and 1:20, or between 1:100 and 1:50.

In another aspect, a method of delivering a composition described herein to a mammal can include orally dosing the composition to the mammal. In certain circumstances, orally dosing the composition can include spraying the composition onto at least a portion of the tongue of the mammal, spraying the composition under at least a portion of the tongue of the mammal or depositing a drop of the composition onto at least an oral mucosal membrane of the mammal.

In certain aspects, the composition can include a plant based additive. In certain embodiments, the plant based additive can include an oil, an extract, for example, a whole plant extract, or combinations thereof. In general, the composition can include no more than 5 wt % of the plant based additive. The plant based additive can be a terpene, flavonoid, polyphenolic, tumeric, ginger, lavender, cumin, valerian root, chamomile, arnica, humulene, white willow bark, or cinnamon.

Other aspects, embodiments, and features will be apparent from the following description, the drawings, and the claims.

### DETAILED DESCRIPTION

Pure cannabinoid and a pure triglyceride can be combined to form a composition with unexpectedly advantageous properties, which are described below. Importantly, it is the combination of a pure cannabinoid, for example with a purity of at least 98%, with a pure caprylic acid triglyceride, for example including no more than 2% of a carboxylic acid other than caprylic acid, that has unexpected properties compared to compositions including broad spectrum cannabidiol and medium-chain triglycerides (MCT). For example, the composition can include at least 70+% C8 triglyceride in combination of at least 98+% pure cannabidiol with the optional addition of 1-3 ingredients such as terpenes, flavonoids or other healthy plant based items such as cumin or cinnamon.

Cannabidiol can be sourced from plants. For example, hemp is high in cannabidiol (CBD), and low in tetrahydrocannabinol (THC), which is psychoactive. Marijuana is the opposite. THC has psychoactive properties and CBD has therapeutic qualities.

Using CBD has been shown to reduce anxiety and depression in both human and animal studies. CBD, especially in combination with THC, may be effective in reducing pain associated with diseases like multiple sclerosis and rheumatoid arthritis. Though CBD has been shown to help reduce symptoms related to cancer and cancer treatment, and may even have cancer-fighting properties, more research is needed to assess its efficacy and safety.

CBD may have beneficial effects on acne due to its anti-inflammatory qualities and its ability to control the overproduction of sebum from the sebaceous glands. Research also shows that CBD accelerates muscle recovery after exercise, and is becoming popular in the athletic community due to the same anti-inflammatory qualities.

Though research is limited at this time, CBD has been shown to effectively treat symptoms related to epilepsy and Parkinson's disease. In fact, FDA just approved the first CBD related drug for epilepsy. CBD was also shown to reduce the progression of Alzheimer's disease in test-tube and animal studies. Though more human studies are needed, CBD may benefit heart health in several ways, including by reducing blood pressure and preventing heart damage.

CBD processing relies primarily on first extracting the plant material and then isolating the extracts and components via evaporation and distillation. Examples of steps to purify CBD are described herein.

One method of purifying CBD is cold ethanol extraction. This process can involve chilling ethanol to less than about −20° C. (−4° F.) either in a cold room or freezer and then pumping the chilled ethanol into a container of cannabis. After a soak period, the plant material is removed in a ‘tea bag’ fashion, for example, by filtration. The ethanol-soaked cannabis is then concentrated by removing the ethanol using rotary evaporators. About 97% of the ethanol is recovered in the process. The extraction takes about two to four hours, yielding an oil-like mixture that contains plant lipids, waxes, fats, terpenes, as well as CBD and other cannabinoids. The mixture can then be cooled, chilling to temperatures less than about −20° C. which causes the lipids, fats and waxes to precipitate. This cooling process can be conducted with jacketed vessels that are cooled through recirculating chillers. This oil is known as full spectrum oil because it contains other cannabinoids and terpenes besides CBD. The resulting product is about 65% pure CBD. Most hemp or CBD products on the market are marketed as full spectrum and broad spectrum oil (see extraction methods above). However, no full spectrum batch is the same. Just as wine, even when the plant is grown in the same exact location, climate, soil and other variables can cause the plant to differ from harvest to harvest, this is a major weakness of using full spectrum oil.

A higher purity CBD can be obtained by CO₂ extraction. CO₂ extraction machines use cold temperatures (less than −60 F), pressure (1,500 to 2,000 psi) and a closed loop to extract oil from the cannabis flower. Because the solubility in CO₂ varies with pressure, CO₂ can be used to extract selected compounds (rather than a combination of all compounds) which makes it more efficient for making consistent, specific formulations like a CBD isolate powder. After extraction, the oil is an amber color but terpenes have been lost in the process and need to be re-introduced. When terpenes are re-introduced, the oil is about 80% pure and known as broad spectrum oil.

An even higher purity CBD can be obtained by distillation. Isolating CBD by this method requires use of a short path distillation apparatus. The oil is heated in a flask under vacuum (typically with a magnetically stirred hot plate) with a short path distillation attachment. The condenser is cooled with a recirculating chiller to provide cooling for condensation of the component vapors. As the vapor temperature increases, indicating a new compound or mixture fraction, the multi-position receiving flask can be adjusted to isolate the different fractions. The CBD oil obtained in this way is now clear, is about 90% pure, and is known as CBD distillate. This oil is popular in VAPE pens and edible because it can be flavored and measured consistently at this level of purity.

The highest purity CBD is known as an isolate. The clear oil, or distillate, goes through another purification process, where it is converted into powder using a solvent, for example, hexane, at low heat. A glass like product is obtained through this extraction method, which can then be broken up and “powderized.” This powder is about 99% pure CBD, but still retains the quality of an oil, meaning that the CBD is not water soluble.

By using these methodologies or comparable methodologies, a cannabidiol of sufficient purity for the composition described herein can be obtained. The cannabidiol can be a pure cannabidiol. For example, the cannabidiol can be of at least 80%, at least 85%, at least 95% purity, at least 97% purity, at least 98% purity, or at least 99% purity. In certain embodiments, the purity is 95% or greater.

Prior to preparing the compositions described herein, other CBD products were made, which include food products, including beverages, and mixtures with multiple components, such as an oil/liquid form that is taken directly from a dropper. The products can be flavored. A CBD isolate powder, which is a high purity CBD, can have powerful, and instant benefits. A water soluble CBD/vitamin E mixture can be included in food and drink such as cold pressed juice. The liquid CBD did not perform as expected, but it was more bio-available than the powder, allowing a decrease in dosage from 50 mg to 20 mg. Treatments with the powder exhibited anti-inflammatory properties, particularly relieving stiffness in joints. However, the powder was difficult to work with and dose.

In certain embodiments, the CBD can be combined with, or replaced with THC. The THC has the purity comparable to the CBD.

By mixing with fractionated coconut oil, or medium-chain triglyceride (MCT), a stable emulsion can be formed, which simplifies oral administration of CBD. Some products can mix the full spectrum CBD oil with alcohol or MCT oil, which is known as a tincture. For example, a CBD isolate can be mixed with MCT oil at 150 degrees to form an oil blend that remains emulsified at both hot and cold temperatures. The emulsion with MCT had increased bioavailability, similar to the vitamin E mixture, because of the way it is accepted into the bloodstream and metabolized by the liver. For example, a batch of CBD/MCT oil was made using a ratio of 100 mg/ml, which allowed delivery as a spray to the mouth of 20 mg CBD per spray (one spray per serving). In another experiment, a second batch of oil targeted at 33 mg/ml, which targeted 6-7 mg per spray (2-3 sprays per serving). Performance can continue to be less impressive than the CBD powder. Another composition contained pure caprylic acid triglyceride and pure cannabidiol (C8/CBD) at 50 mg CBD per ml of C8 that can be delivered to the mouth of a mammal as a spray containing about 9-11 mg per spray.

Alternative dosage sizes, different pumps/bottle types, and alternatives to MCT oil can be explored. MCT oil is generally available in large quantities from environmentally responsible sources. However, not all MCT oils are the same. In general, MCT oil is a triglyceride made up of several types of medium length fatty acids esters with glycerol. The medium length fatty acids have six to fourteen carbons in length, particularly C8, C10 and C12 chains. MCT oil contains a mixture of caprylic acid (C8), capric acid (C10) and lauric acid (C12), each of which has own unique health attributes and benefits. Most MCT oil products are generally a mixture of caprylic acid (C8) and capric/decanoic acid (C10) and sometimes lauric acid (C12). The exact ratio of each varies by MCT oil source and typically contains between 50 to 75% caprylic acid (C8). The ratio of C8, C10 and C12 in the oil can make a difference in compositions delivering CBD, particularly pure cannabidiol. The C8 chain is one of the least available for extraction which is why it is more difficult and costly to acquire from coconuts or palms. There is a very small supply of C8 in the marketplace and its cost and availability cause most products to use the combination of C8/C10.

The composition can be used to treat subjects with appetite issues, for example, due to cancer, to alleviate chronic pain, to relieve epilepsy, to treat movement problems associated with Huntington's Disease, to regulate sleep disorders, to minimize multiple sclerosis symptoms, to help manage schizophrenia, or to provide relief in glaucoma. The daily dosages can be 1 mg, 2.5-20 mg, 200-300 mg, 10 mg per pound of weight, 40-160 mg, 2.5-120 mg, or 40-1,280 mg, respectively.

In other examples, up to 20mg of CBD per kilogram among children and adults produced positive results for severe epilepsy. Pain can be managed with 5-25 mg per day. Anxiety can be managed with 5-25mg per day. Sleep conditions can be managed with 12-25mg per day.

In one aspect, the discovery described herein is a composition including primarily caprylic acid triglyceride (“C8”). In another aspect, the discovery described herein is an oral spray application of a composition including C8 in combination with either a single or combination of cannabinoids with either a single or combination of other plant based additives i.e. terpenes, flavanoids, cinnamon, or cumin.

The plant based additive can be an extract or an oil. In certain embodiments, the extract can be a whole plant or a portion of a plant (i.e., leaf or flower) steeped in an oil, preferably, C8 triglyceride.

The cannabinoid can be, for example, THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), or CBT (cannabicitran). The cannabinoids, particularly pure cannabinoids, can be single compounds in the composition or combined in to have efficacy for particular health benefits. For example, combinations of cannabinoids include THC and THCA, THC and CBD, THC and CBDA, THC and THCV, THC and CBDV, THC and CBCV, THC and CBGV, THC and CBGM, THC and CBE, THC and CBT, THCA and CBD, THCA and CBDA, THCA and THCV, THCA and CBDV, THCA and CBCV, THCA and CBGV, THCA and CBGM, THCA and CBE, THCA and CBT, CBD and CBDA, CBD and THCV, CBD and CBDV, CBD and CBCV, CBD and CBGV, CBD and CBGM, CBD and CBE, CBD and CBT, CBDA and THCV, CBDA and CBDV, CBDA and CBCV, CBDA and CBGV, CBDA and CBGM, CBDA and CBE, CBDA and CBT, THCV and THCV, THCV and CBDV, THCV and CBCV, THCV and CBGV, THCV and CBGM, THCV and CBE, THCV and CBT, CBDV and CBCV, CBDV and CBGV, CBDV and CBGM, CBDV and CBE, CBDV and CBT, CBCV and CBGV, CBCV and CBGM, CBCV and CBE, CBCV and CBT, CBGV and CBGM, CBGV and CBE, CBGV and CBT, CBGM and CBE, CBGM and CBT, or CBE and CBT. The secondary cannabinoid can be 2 to 15 wt % of the cannabinoid content. The C8 cannabinoid compositions can include terpenes, flavonoids or other plant based elements. The C8 can increase bioavailability of components compared to MCT compositions.

Various cannabinoids can have the following health effects:

CBD: pain relieving, antioxidant, anti-inflammatory, anti-seizure and anti-nausea

CBDA: precursor to cannabidiolate and acts as an enzyme which synthesizes cannabidiol

CBDV: antiepileptic and anticonvulsant properties

CBC: anti-inflammatory, pain relieving and antidepressant properties, anti-fungal and anticonvulsant properties

CBG: antidepressant, antianxiety and anti-inflammatory effects

CBN: anticonvulsant and anti-inflammatory effects

THC: psychoactive properties

THCV: demonstrated anti-obesity effects, management of type II diabetes

The plant based additives can include terpenes. The terpene content can be 0.5 to 8 wt % of the composition. The terpene can have the following health effects.

Terpene Chart

| | | | | | |
|---|---|---|---|---|---|
| Limonene | Linalool | Caryophyllene | Humulene | Myrcene | Pinene? |
| Anxiety | Anxiety | Inflammation | Inflam- | Inflam- | Inflam- |
| | | | mation | mation | mation |
| AntiFungal | Pain | Pain | Pain | Antifungal | Asthma |
| GI Reflux | Insomnia | Insomnia | Anti- | Anti- | |
| | | | Bacterial | Bacterial | |
| Depression | Depression | Antioxidant | | Antiseptic | |
| | Stress | Muscle | | | |
| | | Spasms | | | |
| | Convulsions | | | | |

Unexpectedly, the caprylic acid triglyceride forms a composition with pure cannabidiol that has unexpectedly beneficial properties described below. Triglycerides with a C8 chain can provide immediate energy because it can bypass the stomach and transport more directly into the bloodstream. MCT oils enhances in C8 content can be beneficial because of the C8 oil can be found in ketogenic diets. Research has shown that caprylic acid (C8) ingestion can increase ketone body production significantly. Even a modest increase of 1 mMol in plasma caprylic acid levels can increase ketone body production five-fold. Moreover, despite the presence of moderate amounts of blood glucose (such as when you've been eating carbohydrates or higher amounts of protein), caprylic acid is still readily absorbed from the gastrointestinal tract and rapidly oxidized. This means the C8 is converted into ketones regardless of whether a mammal is in ketosis and following a ketogenic diet or not. Other research has demonstrated that caprylic acid does indeed have strong antibacterial, antiviral, and antifungal activities, and has also been shown to improve blood lipid profiles of hypertensive rats, though data in humans remains limited.

The pure triglyceride is a triglyceride containing glycerol and substantially a single chain length carboxylic acid. By substantially a single chain length, less than 10%, preferably less than 5% or more preferably less than 2% of the carboxylic acid component of the triglyceride is of a chain length different from the predominant chain length of the triglyceride. For example, the pure triglyceride can be a C8 triglyceride, such as caprylic acid triglyceride containing less than 10%, less than 5% or less than 2% of a capric acid component and at least 90%, at least 95%, or at least 98% of a caprylic acid component. Caprylic acid, or octanoic acid, and caprylic acid triglyceride can be isolated from coconut oil, palm kernel oil or milk from mammals.

Pure caprylic acid triglyceride, e.g., containing less than 2% of a capric acid component and at least 98% a caprylic acid, can be used to formulate a composition with pure cannabidiol. One composition contained pure caprylic acid triglyceride and pure cannabidiol (C8/CBD) at 33 mg CBD per ml of C8 that can be delivered to the mouth of a mammal as a spray containing about 6-7 mg per spray. In another experiment, a second batch of oil targeted at 50 mg/ml, which targeted about 9-11 mg per spray (1-2 sprays per serving). After giving this composition to test subjects, unexpected feedback was received. For example, bioavailability and efficacy improved significantly. This C8/CBD mixture only required one spray to make a difference and users noticed its effect almost immediately. The C8/CBD composition was approximately three times more effective than a comparable composition using MCT instead of caprylic acid triglyceride. Other subjects who had never felt effects of CBD before (either in powder or in the regular MCT), now benefited from the CBD; reducing their anxiety, providing ability to fall asleep faster and deeper. Users of CBD powder that tried the C8/CBD spray recognized similar effects. Treatment with the composition was significantly more effective for their anxiety. Most significantly, the C8/CBD test users started recommending it to their friends.

The weight ratio of cannabidiol to triglyceride in the composition can be between 1:1 and 1:6, between 1:3 and 1:5, or around about 1:4. The composition can include 0.01 wt % to 40 wt % cannabidiol and 90 wt % to 99.99 wt % caprylic acid triglyceride. In certain circumstances, the composition can include at least 70 wt % caprylic acid triglyceride in combination with a pure cannabidiol. The composition can contain 5 mg CBD/ml, 10 mg CBD/ml, 12 mg CBD/ml, 15 mg CBD/ml, 20 mg CBD/ml, 25 mg CBD/ml, 30 mg CBD/ml, 33 mg CBD/ml, 35 mg CBD/ml, 40 mg CBD/ml or 50 mg CBD/ml.

In certain circumstances, the composition can include one or more plant based additives. For example, the composition can include a terpene, flavonoid, a polyphenol, tumeric, ginger, lavender, cumin, valerian root, chamomile, arnica, humulene, white willow bark, or cinnamon or other phytochemicals. For example, tumeric, ginger, lavender, chamomile, arnica, valerian root, humulene, white willow bark, and cumin can provide therapeutic benefits as well. A whole plant, flower or herb can be steeped in C8 triglyceride, a Caprylic extract oil can be obtained. The Caprylic extract oil can be Caprylic Camomile oil when camomile plant is steeped in C8 triglyceride. The steeping can occur at 100 to 300 degrees F. for up to 48 hours. For example, the steeping can occur for up to 24 hours, up to 36 hours or up to 48 hours. The plant based additive can include an oil, an extract, for example, a whole plant extract, or combinations thereof.

The composition can be delivered to a mammal by various methodologies. For example, the composition can be delivered orally as a liquid. The liquid can be placed on a tongue with a dropper, can be placed under a tongue with a dropper, placed on the tongue as a spray, placed under the tongue as a spray, or placed on a cheek as a spray. The spray can deliver a mist over a broad area facilitating absorbance and bioavailability of the composition to the mammal. Advantageously, this mode of delivery can be more reliable and uniform. For example, dosing by spray can be more measurable. A full dropper is 0.8 ml or roughly 25 mg of CBD in a 33 mg/ml mixture. However, a typical dose can be 10 to 12 mg, which can require delivery of half dropper amounts which can be difficult to measure. The composition can be prescribed as doses in drops, i.e., four to eight drops, or spray pumps. Spraying a fine mist orally especially under the tongue reaches oromucosal receptors in the mouth more efficiently than other methods. This method of dosing can be more efficient, leading to less waste. This method of dosing can also be more discreet, particularly compared to vapor delivery methodologies, such as vaping.

Unexpectedly, the composition of pure caprylic acid triglyceride and pure cannabidiol can be more bioavailable compared to MCT oil compositions. The caprylic acid triglyceride compositions are less viscous and absorbed faster.

Formulation examples follow:

Formulation Examples of Cannabinoid, Terpenes and Other
Plant Based Additives

Anti-Anxiety
CBD, CBG, Limonene, Linalool
Anti-Depression
CBC, CBG, Limonene, Linalool
Inflammation
CBD, CBDA, CBC, CBG, CBN, Caryphyllene, Humulene, Myrcene, Pinen
Pain
CBD, CBC, Linalool, Caryophyllene, Humulene
Anticonvulsant
CBD, CBDV, CBC, CBN, Linalool, Caryophyllene
Insomnia
CBD, Linalool, Caryophyllene, Chamomile
Or
CBD, Capyrlic Chamomile, Valerian Root
Formulation Examples in milligrams (mg)
In mgs

| | | Composition | Composition | Composition |
|---|---|---|---|---|
| | Anti-Anxiety | 1 | 2 | 3 |
| | C8 | 30,000 | 30,000 | 30,000 |
| Cannabinoid | CBD | 1,500 | 1,500 | 1,500 |
| Cannabinoid | CBG | 30 | 150 | 225 |
| Terpene | Limonene | 8 | 60 | 120 |
| Terpene | Linalool | 120 | 8 | 60 |
| | In mgs | | | |
| | Anti- | Composition | Composition | Composition |
| | Depression | 1 | 2 | 3 |
| | C8 | 30,000 | 30,000 | 30,000 |
| Cannabinoid | CBC | 1,500 | 750 | 30 |
| Cannabinoid | CBG | 30 | 750 | 1,500 |
| Terpene | Limonene | 8 | 30 | 120 |
| Terpene | Linalool | 120 | 30 | 8 |
| | In mgs | | | |
| | | Composition | Composition | Composition |
| | Inflammation | 1 | 2 | 3 |
| | C8 | 30,000 | 30,000 | 30,000 |
| Cannabinoid | CBD | 1,500 | 1,500 | 30 |
| Cannabinoid | CBDA | 30 | 150 | 1,500 |
| Cannabinoid | CBC | 31 | 165 | 230 |
| Cannabinoid | CBG | 31 | 165 | 230 |
| Terpene | Caryophyllene | 7.65 | 122 | 122.4 |
| Terpene | Humulene | 15 | 7 | 70 |
| Terpene | Myrcene | 122 | 15 | 8 |
| | In mgs | | | |
| | | Composition | Composition | Composition |
| | Insomnia | 1 | 2 | 3 |
| | C8 | 30,000 | 30,000 | 30,000 |
| Cannabinoid | CBD | 1,500 | 1,500 | 1,500 |
| Terpene | Linalool | 120 | 8 | 60 |
| Terpene | Caryophyllene | 8 | 60 | 120 |
| Plant Based | Chamomile | 35 | 7 | 55 |

Users of the composition of pure caprylic acid triglyceride and pure cannabidiol unexpected report the following:
Anxiety decreased in 40 year old woman, when compared to isolate
Anxiety decreased in 16 year old male, when compared to 100 mg/ml MCT spray
Sleep improved in 65 year old female, when compared to 33 mg/ml MCT spray
Sleep improved in 16 year old male, when compared to 33 mg/ml MCT spray
Anxiety reduced in 40 year old male, when compared to 33 mg/ml MCT spray
Anxiety reduced in 47 year old male, when compared to 33 mg/ml MCT spray
Mood stabilized in 38 year old male when compared to 33 mg/ml MCT spray
Mood stabilized in 36 year old female when compared to 33 mg/ml MCT spray

### EX 1

### Sleep Formula

C8 was steeped in Chamomile for 24-48 hours at between 100 and 300 degrees Fahrenheit which produced Caprylic Chamomile oil. CBD and valerian root were then added.

| | | Circumstance | Circumstance | Circumstance |
|---|---|---|---|---|
| | Sleep Aid | 1 | 2 | 3 |
| | Caprylic | 30,000 | 30,000 | 30,000 |
| | Chamomile Oil, | | | |
| | C8/Chamomile | | | |
| Cannabinoid | CBD | 1,500 | 1,500 | 1,500 |
| Plant Based | Valerian root | 120 | 8 | 55 |

### EX 2

### Pain Formula

C8 was steeped in Arnica and White Willow Bark for 24-48 hours at between 100 and 300 degrees Fahrenheit which produced Caprylic Arnica and White Willow Bark Oil. CBD, Humulene and Mullein were then added to the Caprylic Arnica and White Willow Bark Oil.

| | | Circumstance | Circumstance | Circumstance |
|---|---|---|---|---|
| | Insomnia | 1 | 2 | 3 |
| | Caprylic Arnica | 30,000 | 30,000 | 30,000 |
| | and White | | | |
| | Willow Bark | | | |
| | Oil, C8/Arnica | | | |
| | and White | | | |
| | Willow Bark | | | |
| Cannabinoid | CBD | 1,500 | 1,500 | 1,500 |
| Terpene | Humulene | 160 | 12 | 49 |
| Plant-Based | Mullein | 10 | 75 | 114 |

Details of one or more embodiments are set forth in the accompanying drawings and description. Other features, objects, and advantages will be apparent from the description, drawings, and claims. Although a number of embodiments of the invention have been described, it will be understood that various modifications may be made without departing from the spirit and scope of the invention. It should also be understood that the appended drawings are not necessarily to scale, presenting a somewhat simplified representation of various features and basic principles of the invention.

1. A composition comprising:
60 wt % to 99.99 wt % caprylic acid triglyceride.

2. The composition of claim 1, wherein composition includes 90 wt % to 99.99 wt % caprylic acid triglyceride.

3. The composition of claim 1, wherein composition includes 0.01 wt % to 40 wt % cannabidiol.

4. The composition of claim 3, wherein the cannabidiol is of at least 95% purity.

5. The composition of claim 3, wherein the cannabidiol is of at least 97% purity.

6. The composition of claim 3, wherein the cannabidiol is of at least 98% purity.

7. The composition of claim 3, wherein the cannabidiol is of at least 99% purity.

8. The composition of claim 1, further comprising a terpene, flavonoid, polyphenolic, tumeric, ginger, lavender, cumin, valerian root, chamomile, arnica, humulene, white willow bark, or cinnamon.

9. A composition consisting essentially of a cannabinoid, caprylic acid triglyceride and, optionally, 1, 2, 3, 4 or 5 different plant based additives.

10. The composition of claim 9, wherein the weight ratio of cannabinoid to caprylic acid triglyceride is between 1:500 and 1:10.

11. The composition of claim 9, wherein the weight ratio of cannabinoid to caprylic acid triglyceride is between 1:250 and 1:20.

12. The composition of claim 9, wherein the composition has up to 5wt % of the plant based additive.

13. The composition of claim 9, wherein the plant based additive is a terpene, flavonoid, polyphenolic, tumeric, ginger, lavender, cumin, valerian root, chamomile, arnica, humulene, white willow bark, or cinnamon.

14. The composition of claim 9, wherein the cannabinoid includes one or more of cannabinol, cannabidiol, dronabinol (delta-9-tetrahydrocannabinol), delta-8-tetrahydrocannabinol, 11-hydroxy-tetrahydrocannabinol, 11-hydroxy-delta-9-tetrahydrocannabinol, levonantradol, delta-11-tetrahydrocannabinol, or tetrahydrocannabivarin.

15. A composition comprising:
0.01 wt % to 40 wt % tetrahydrocannabinol; and
60 wt % to 99.99 wt % caprylic acid triglyceride.

16. The composition of claim 15, wherein composition includes 90 wt % to 99.99 wt % caprylic acid triglyceride.

17. The composition of claim 15, wherein composition includes 0.01 wt % to 10 wt % tetrahydrocannabinol.

18. The composition of claim 15, wherein the tetrahydrocannabinol is of at least 95% purity.

19. The composition of claim 15, wherein the tetrahydrocannabinol is of at least 97% purity.

20. The composition of claim 15, wherein the tetrahydrocannabinol is of at least 98% purity.

21. The composition of claim 15, wherein the tetrahydrocannabinol is of at least 99% purity.

22. The composition of claim 15, further comprising a terpene, flavonoid, polyphenolic, tumeric, ginger, lavender, cumin, valerian root, chamomile, arnica, humulene, white willow bark, or cinnamon.

23. A composition consisting essentially of cannabidiol, tetrahydrocannabinol or a combination thereof, caprylic acid triglyceride and, optionally, 1, 2, 3, 4 or 5 different plant based additives.

24. The composition of claim 23, wherein the weight ratio of tetrahydrocannabinol to caprylic acid triglyceride is between 1:500 and 1:10.

25. The composition of claim 23, wherein the weight ratio of tetrahydrocannabinol to caprylic acid triglyceride is between 1:250 and 1:20.

26. The composition of claim 23, wherein the plant based additive is a terpene, flavonoid, polyphenolic, tumeric, ginger, lavender, cumin, valerian root, chamomile, arnica, humulene, white willow bark, or cinnamon.

27. A method of delivering the composition of claim 1 to a mammal comprising:
orally dosing the composition to the mammal.

28. The method of claim 27, wherein orally dosing the composition includes spraying the composition onto at least a portion of the tongue of the mammal.

29. The method of claim 27, wherein orally dosing the composition includes spraying the composition under the tongue of the mammal.

30. The method of claim 27, wherein orally dosing the composition includes depositing a drop of the composition onto at least an oral mucosal membrane of the mammal.
